(19) United States
(12) Patent Application Publication
(10) Pub. No.: US 2007/0293888 A1
(43) Pub. Date: Dec. 20, 2007

(54) Self-Closing External Vascular Closure

(76) Inventors: Ernst-Diethelm Harren, Steinhausen (CH); Marc Ulrich Lehmann, Herrliberg (CH)

Correspondence Address: WILLIAM COLLARD;COLLARD & ROE, P.C., 1077 NORTHERN BOULEVARD, ROSLYN, NY, 11576, US

(21) Appl. No.: 11/665,936

(22) PCT Filed: Oct. 18, 2005
(86) PCT No.: PCT/CH05/00609

§ 371 Date: May 8, 2007

(30) Foreign Application Priority Data
Oct. 20, 2004 (CH) 1731/04

(51) Int. Cl. A61B 17/03

(52) U.S. Cl. 606/201

(57) ABSTRACT

A self-closing external vascular closure (1) for closing an arterial or venous blood vessel having a puncture opening in a human or animal body by means of autologous blood has a pressure chamber (6) that can be attached to the body in the area of the puncture opening and can be put under an excess pressure, the pressure chamber having a closure part (5) made of silicone designed in one piece with an integrated closure element (7) in its area facing away from the body. The pressure chamber (6) has a pressure wall part (2) which is also designed in one piece in its area facing the body. The self-closing external vascular closure (1) is adhesively attachable, transparent and puncturable by a needle, cannula, catheter or the like. The pressure part wall (2) with offshoots (9) extends over the entire area of the external vascular closure (1) that can be adhesively attached to the body, and an adhesive layer (4) is attached to a large area between the pressure wall part (2) and the closure part (5), excluding the pressure chamber area. The advantages include a very good adaptability to different topographies of skin.

The invention relates to a self-closing vascular closure for closing an arterial or venous blood vessel having a puncture opening in a human or animal body using autologous blood according to Patent claim 1. Additional applications also include arteriovenous shunts, prostheses and the like.

The invention relates in particular to such a vascular closure with a pressure chamber that can be put under excess pressure and can be secured on the body in the area of the puncture opening, whereby this pressure chamber is filled with blood in the case of a puncture for the purpose of hemostasis by creating a pressure equilibrium with the blood flowing out of the blood vessel.

There is still a demand, especially in clinical and polyclinical facilities, for self-closing external vascular closures that are simple to produce and easy to handle while having a reliable effect for closing a puncture opening by means of autologous blood.

EP 0 955 901 discloses a tight puncture closure of this type in two embodiments.

The first embodiment according to EP 0 955 901 has an external puncture closure for closing an arterial blood vessel having a puncture opening in a human or animal body using autologous blood, with a pressure chamber that can be attached to the body in the area of the puncture opening and can be put under excess pressure, said pressure chamber having a closure part designed in two parts in the area facing away from the body and consisting of a retaining wall made of polyether urethane film, a polyether film or a polypropylene film and having a closure element made of silicone. The retaining wall and the closure part may be glued together. The pressure chamber has a pressure wall part designed in one piece in the area facing the body.

This external vascular closure is attachable adhesively, is transparent and is puncturable by a needle or a cannula or the like.

However, there are disadvantages when the retaining wall and the closure part are manufactured separately and then must be joined together (by gluing or welding), which necessitates a greater manufacturing complexity. However, the connection of the pressure wall part to the retaining wall may be created by gluing using a silicone adhesive or in the case when identical materials are used for the pressure wall part and the retaining wall, the connection may also be formed by welding.

A second embodiment according to EP 0 955 901 has an external vascular closure for sealing an arterial blood vessel having a puncture opening in a human or animal body using autologous blood with a pressure chamber that can be attached to the body in the area of the puncture opening and can be put under excess pressure, said pressure chamber having a thick closure part made of silicone designed in one piece with an integrated closure element in the area facing away from the body. The pressure chamber has a pressure wall part designed in one piece in its area facing the body.

This external vascular closure is also attachable adhesively, is transparent and is puncturable by a needle or cannula.

However, there are disadvantages because the thickness of the closure part makes it impossible for the arterial puncture closure described here to be readily adaptable to the local topography of the skin. Furthermore, the adhesive layer that is provided and is in direct contact with the skin must also have good properties of being tolerated well by the skin, which is in turn not readily applicable to adhesives that are suitable for use with silicone. Furthermore, there is the risk of separation because of the small area of the adhesive bond between the closure part and the pressure wall.

On the whole, the problem of the tolerability of silicone adhesives by the skin is not addressed with either of the two embodiments according to EP 0 955 901.

The object of the present invention is to provide an improved self-closing external vascular closure that is especially simple to manufacture.

This object is achieved by the features of Patent claim 1.

With a generic self-closing external vascular closure with a closing part made of silicone and designed in one piece with an integrated closing element and with a pressure wall part that is likewise designed in one piece, the inventive solution consists of the fact that the pressure wall part is designed so that it extends with offshoots over the entire area that is to be attached to the body with adhesive, and furthermore since the pressure part wall is entirely provided with a skin-adhesive layer that is tolerated especially well by the skin, and an adhesive layer that is applied between the pressure wall part and the closure part over a large area, not including the pressure chamber area. The aforementioned adhesive layer need not have any special properties of being tolerated well by the skin because it does not come in contact with the skin, and furthermore, it is left free over the entire puncture area. Instead, it may be selected in such a way as to result in optimal adhesive properties with regard to the materials to be joined, which usually requires special adhesives and pretreatments in the case of silicone.

It is also possible to provide for the adhesive layer, which in the simplest case consists only of an adhesive (also known as an industrial adhesive, i.e., an adhesive without the specific properties of a skin-adhesive) has a backing film that is provided with adhesive on both sides. Such a structure of the adhesive layer may be selected to simplify production in the sense that the adhesive layer may also be designed as a “vendor part” for the production of the self-closing external vascular closure.

The closure element integrated into the closure part is preferably designed like a segment of a sphere or with a lenticular shape, round or elliptical. The closure part is preferably at least 4 mm thick in the area of the integrated closure element. Therefore, the external vascular closure can be exposed to excess pressure especially easily.

A good and secure fastening of the external vascular closure to a wide variety of topographies of skin can be achieved by the fact that the self-closing external vascular closure is provided with a number of soft and thin offshoots. The inventive self-closing external vascular closure consists of only two layers of material, in the offshoots as well, namely an upper layer shaped from the closure part designed in one piece, and a lower layer shaped from the pressure wall part, which is also designed in one piece, so this yields optimal adaptation possibilities with regard to the mechanical requirements and does so in several regards. First, there is the secure adhesion of the external vascular closure to the skin in a manner that is tolerated well by the skin, while on the other hand, there is the secure and reliable adhesion of the closure part made of silicone to the pressure wall part. Since the closure part also assumes the function of a stabilization wall in the area of the integrated closure element in the form of segments of a sphere for the purpose of building up a backpressure, it is therefore also necessary to ensure that the pressure chamber which fills up with blood flowing out of the vessel will develop in the direction of the interior of the body. Therefore, the reliability of the adhesive bond and the combination of materials is especially important also in the transitional areas between the offshoots and the pressure chamber. This is all the more true because, although silicone has the desired reclosing properties after being punctured with a cannula or needle, at the same time it also has an extremely high extensibility. According to this invention, the result achieved with the resulting freedom of choice for a silicone adhesive that is actually reliable is that the adhesive bond does not become loosened in the transitional areas at the suddenly occurring pressure peaks. To further improve the adhesive bond, it is also possible to provide for the silicone of the closure part to be subjected to a plasma treatment, a corona treatment, a wet chemical treatment or some other treatment on the adhesive layer side before forming the adhesive bond.

With regard to the combination of materials between the closure part made of silicone and the pressure wall part, it has proven to be advantageous to also use for the latter a soft material that adapts well to the topography of the skin and has an extensibility that is high but is usually lower than that of silicone. The material for the pressure wall part must be able to form an extensible pressure wall in the pressure chamber area facing the puncture opening. The pressure wall part is preferably made of a 5 μm to 50 μm thick, but preferably approx. 25 μm thick polyether urethane film, polyether film or polypropylene film.

A skin-adhesive layer that has one or more of the properties from the following group of properties due to the addition of corresponding active ingredients is preferably also selected:
antiseptic properties,
antiallergic properties,
analgesic properties.

The inventive self-closing external vascular closure according to this invention is described in greater detail below on the basis of an exemplary embodiment with drawings.

In the drawings:

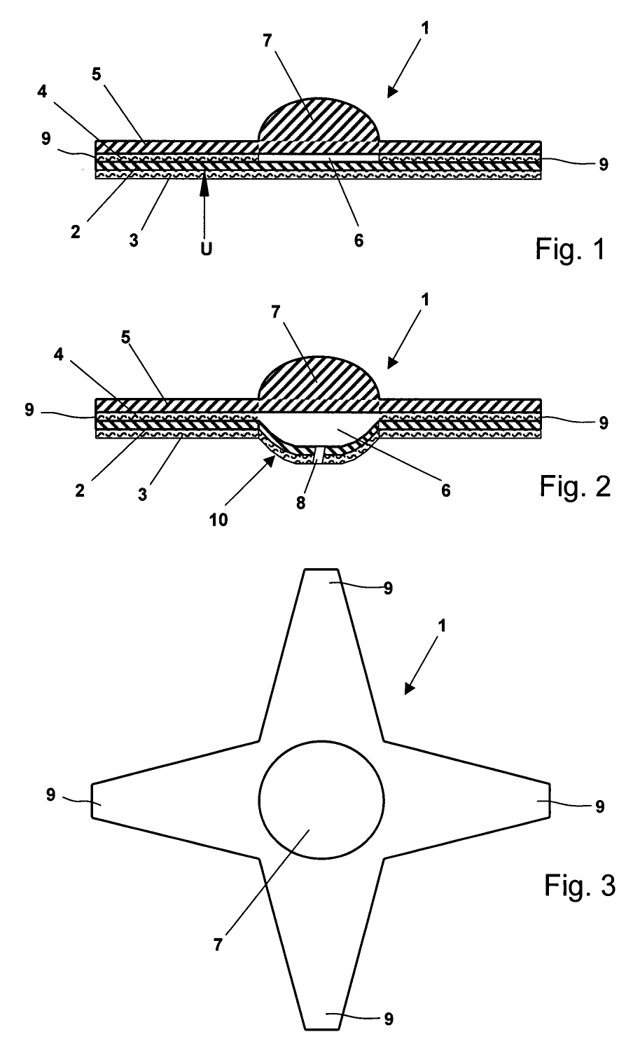

FIG. 1 shows a cross section through a self-closing external vascular closure in the condition before puncturing a blood vessel,

FIG. 2 shows another cross section through a self-closing external vascular closure according to FIG. 1 in the condition after closure of the puncture of a blood vessel, and

FIG. 3 shows a top view of a self-closing external vascular closure according to FIG. 1.

FIG. 1 shows a cross section (not drawn to scale) through a self-closing external vascular closure 1, whereby the vascular closure 1 is shown in the condition prior to puncturing a blood vessel.

A one-piece pressure wall part 2, usually thin (like a film) is provided with adhesive layers on both sides. A (medicinal) skin-adhesive layer 3 is provided on an underside U of the pressure wall part 2, this side of the self-closing external vascular closure 1 being provided for being attached adhesively to human skin. On the opposite side of the pressure wall part 2, there is an (industrial) adhesive layer 4. The adhesive layer 4 is provided between the pressure wall part 2 and a closure part 5, also designed in one piece, and does not have any specific skin-adhesive properties. The closure part 5 is preferably made of silicone. However, the only adhesives currently known for silicone have little or no good tolerability on skin. With the intended structure of the self-closing external vascular closure 1, the adhesive layer 4 need not have any specific properties as a skin-adhesive and/or in particular need not have skin-friendly properties because it does not come in contact with skin. The adhesive layer 4 also does not come in contact with a cannula, a needle or the like because the adhesive layer 4 is excluded in the puncture area.

The self-closing external vascular closure 1 has a pressure chamber 2 that can be attached to the body in the area of the puncture opening and can be put under an excess pressure. The pressure chamber 6 is situated in an intermediate space between the closure part 5 and the pressure wall part 2 and/or it may develop in this intermediate space. In the area facing away from the body, i.e., on the one-piece closure part 5, the pressure chamber 6 has a closure element 7 that is in the form of a segment of a sphere or has a lenticular, round or elliptical shape and is integrated into the closure part. The pressure chamber 6 in its part facing the body consists only of a partial piece of the thin one-piece pressure wall part 2 that is not glued to the closure part 5. The pressure chamber may also be provided with a coagulating agent and/or a hemostatic agent.

The closure element 7 serves to allow an excess pressure to be applied to the pressure chamber 6 of the self-closing external vascular closure 1 from the outside. The closure part 5 and/or the thickened area of the integrated closure element 7 in the form of a spherical segment, however, also assume the function of a stabilizing wall in the pressure chamber 6 for the purpose of building up a backpressure by ensuring that the pressure chamber 6 which is filling up with blood flowing out [of the vessel] will always develop in the direction of the interior of the body.

All the layers and parts that are used, i.e., the pressure wall part 2, the closure part 5, the adhesive layer 4 and the skin of adhesive layer 3 are preferably transparent or approximately transparent in the entire area of the self-closing external vascular closure 1 or at least in a sufficiently large partial area of the same superimposed upon each other. In addition, soft materials that adapt well to the topography of skin are provided for the pressure wall part 2 and the closure part 5. The pressure wall part 2 is also preferably made of a material that is less extensible than the closure part 5. The greater extensibility of the closure part 5 is thus limited in its areal extent and stabilized, in particular in the case of silicone.

If a layer structure including a backing film and an (industrial) adhesive applied to both sides of the backing film is used for the adhesive layer 4, then one can naturally expect that this backing film, like the pressure wall part 2 and of course also depending on the choice of materials, will contribute toward limiting the extensibility and toward stabilizing the closure part 5.

An especially favorable and preferred combination of materials consists of making the closure part 5 out of silicone and the pressure wall part 2 out of a 5 μm to 50 μm thick, but preferably approximately 25 μm thick polyether urethane film, polyether film or polypropylene film. The closure part 5 is advantageously also at least approximately 4 mm thick in the area of the integrated closure element 7.

FIG. 2 shows another cross section through a self-closing external vascular closure according to FIG. 1, but shown here in the condition after conclusion of the puncture of a blood vessel. The pressure chamber 6 here is filled with blood (not shown). The side of the pressure chamber 6 facing the body has a puncture opening 8 through which the blood could flow into the pressure chamber 6. The side of the pressure chamber 6 facing away from the body respectively the closure element 7 has also been punctured, but the puncture channel has closed again automatically after extraction of the needle or the cannula due to the extremely high restoring force of silicone. The shape of the pressure chamber 6 which is bulging in the direction of the interior of the body as shown here is thus able to develop.

Finally, FIG. 3 shows a top view of a self-closing external vascular closure according to FIG. 1. The self-closing external vascular closure 1 is advantageously provided with a number of soft and thin offshoots 9, so that a good and secure fastening of the self-closing external vascular closure to a wide variety of topographies of skin is achieved.

Except for the actual pressure chamber 6 area (pressure wall 10), i.e., essentially in the area of the offshoots 9, the inventive self-closing external vascular closure 1 thus usually consists of only two layers of material that are glued together over a large area by means of the (industrial) adhesive layer 4, namely the upper layer of material, which is thicker and which is made of the material of the closure part 5 and which is designed in one piece with the integrated closure element 7, and the lower layer of material, which is thinner and which is made of the material of the pressure wall part 2 and which is also designed in one piece and which has the skin-adhesive layer 3 applied to the skin side.

However, it is also possible for the skin-adhesive layer 3 to be applied to the pressure wall part 2 by means of a (film-like) backing layer (not shown) which is usually also thin. In this type of embodiment, another (industrial) adhesive layer is provided (also not shown here) between the pressure wall part 2 and the backing layer. This additional (industrial) adhesive layer can thus readily extend over the entire underside of the self-closing external vascular closure. In this type of embodiment, the backing layer is preferably also made of a material that is less extensible than the closure part 5. Thus the greater extensibility of the closure layer 5, in particular in the case of silicone, is further limited in its areal extensibility and stabilized.

In the embodiment mentioned last, an absorbent material such as a nonwoven or paper may also be used for the backing layer.

In all variants, it is also possible for water-vapor-permeable materials to be used.

FIG. 3 shows as an example an embodiment having four offshoots 9. It is of course also possible to provide another suitable number of offshoots 9. Thus the number of offshoots could be “multiplied” to a shape which covers the area on the whole and is free of offshoots. However, the latter would then tend to make it difficult to securely apply the closure to body areas that are topographically unfavorable. The length of the offshoots 9 of course also has a significant influence on the adhesion ability of the self-closing external vascular closure 1 on the skin and must consequently of course be selected or be ready made in accordance with the intended application.

Use of the self-closing external vascular closure:
The blood vessel intended for puncturing is localized.
The body location to be punctured is cleaned and disinfected in the traditional manner.
At the self-closing external vascular closure 1, the protective films are peeled away and the vascular closure 1 is glued onto the respective body location with the skin-adhesive layer 3. This preserves the antiseptic condition created prior to the puncture before the puncturing.
The vessel intended for puncturing is localized again visually through the transparent surface of the self-closing external vascular closure.
The puncturing is performed by puncturing the external vascular closure, respectively all the layers of the same (except for the adhesive layer 4 which is excluded in the puncturing area) in the area of the closure element 7 with a needle or cannula. In special cases in diagnostic procedures the puncturing is performed by means of a catheter. The intended amount of liquid is removed from and/or supplied to the patient to be treated through the needle or cannula or the diagnostic measure is performed. Since it is always assumed that the procedure is being performed by professional medical personnel, it is also assumed that the syringe or cannula or catheter or other device being used is already sterile. The antiseptic condition is thus also preserved during the puncturing. Because of the elastic restoring force of the closure part 5, it protects the location being punctured from environmental influences even during the procedure because the material is always in tight contact with the needle or cannula. Furthermore, the personnel is protected from possible risks of infection.
After the procedure is performed, the needle or cannula or catheter or the like is removed. Again, the elastic restoring force of the closure part 5 acts in such a manner that the puncture channel in the closure element 7 is sealed again immediately due to the restoring force of the material. The punctured site is thus reliably protected even after the procedure. No microorganisms, no microscopic and/or macroscopic dirt particles and/or viruses and/or bacteria can enter the puncture channel and thus the human or animal body. However, since the puncture opening 8 in the pressure wall 10 remains open after retracting the needle or cannula or the like, blood escaping subsequently can flow through the puncture channel and the puncture opening 8 into the pressure chamber 6 and fill it up. Due to the stabilizing effect of the closure element 7, the pressure chamber 6 (as illustrated in FIG. 2) takes shape in the direction of the interior of the body. As soon as the blood pressure in the pressure chamber 6 corresponds to the pressure of the blood emerging from the puncture channel, the bleeding comes to a standstill.
Due to the design of the self-closing external vascular closure described above, blood escaping from the puncture wound in excess of the filling the pressure chamber is prevented in the normal case. The external vascular closure can at any rate remain on the respective body site as long as necessary or as desired.

### List of Reference Notation

1 external vascular closure
2 pressure wall part
3 (medical) skin-adhesive layer
4 (industrial) adhesive layer
5 closure part
6 pressure chamber
7 closure element
8 puncture opening
9 offshoot
10 pressure wall
U bottom side of the pressure part

1. A self-closing external vascular closure (1) for closing an arterial or venous blood vessel having a puncture opening in a human or animal body by means of autologous blood, having
a pressure chamber (6) that can be attached to the body in the area of the puncture opening and that can be put under excess pressure, having a closure part (5) made of silicone and designed in one piece with an integrated closure element (7) in the area facing away from the body, and the pressure chamber (6) having a pressure wall part (2) designed in one piece in its area facing the body, and
the external vascular closure (1) is adhesively attachable, is transparent and is puncturable by a needle of a cannula, a catheter or the like, wherein
the pressure wall part (2) extends with offshoots (9) on the entire area of the external vascular closure (1) to be attached to the body, and
an adhesive layer (4) is applied between the pressure wall part (2) and the closure part (5) over a large area except for the pressure chamber area.

2. The self-closing external vascular closure (1) according to claim 1,
wherein
the integrated closure element (7) is designed in the form of a segment of a circle or is lenticular, round or elliptical.

3. The self-closing external vascular closure (1) according to claim 1,
wherein
the closure part (5) extends to the offshoots (9), whereby the offshoots (9) are at least 1 mm thick in this area.

4. The self-closing external vascular closure (1) according to claim 1, wherein
the closure part (5) is at least 4 mm thick in the area of the integrated closure element (7).

5. The self-closing external vascular closure (1) according to
claim 1, wherein
the pressure wall part (2) is formed from a soft material that adapts well to the topography of the skin and has an extensibility that is high but is still lower than that of silicone and in particular has an extensible pressure wall (10) in its pressure chamber area facing the puncture opening.

6. The self-closing external vascular closure (1) according to claim 5,
wherein
the pressure wall part (2) is formed from a film of polyether urethane, polyether or polypropylene 5 μm to 50 μm thick, preferably 25 μm thick.

7. The self-closing external vascular closure (1) according to
claim 1, wherein
the adhesive layer (4) need not have any specific properties of a skin-adhesive.

8. The self-closing external vascular closure (1) according to
claim 1, wherein
the adhesive layer (4) has a backing film that is provided with adhesive on both sides.

9. The self-closing external vascular closure (1) according to
claim 1, wherein
there is a skin-adhesive layer (3) which has one or more of the properties from the following group of properties due to active ingredients added:
antiseptic properties,
antiallergic properties,
analgesic properties.

10. The self-closing external vascular closure (1) according to claim 1,
wherein
the skin-adhesive layer (3) is applied to the pressure wall part (2) on the bottom side U or is applied to the bottom side U of the pressure wall part by means of a backing layer and another adhesive layer.

11. the self-closing external vascular closure (1) according to claim 10,
wherein
the backing layer is liquid absorbent.

12. The self-closing external vascular closure (1) according to
claim 1, wherein
the skin-adhesive layer (3) is covered by a removable protective film.

13. The self-closing external vascular closure (1) according to
claim 1, wherein
the closure part (5) is preferably made of silicone pretreated by plasma treatment, corona treatment, wet chemical treatment or some other treatment on the adhesive side.

14. The self-closing external vascular closure (1) according to claim 1, wherein
the water-vapor-permeable materials are used.

15. The self-closing external vascular closure (1) according to
claim 1, wherein
the pressure chamber (6) is provided with a coagulating agent.
